(19) United States
(12) Patent Application Publication
(10) Pub. No.: US 2014/0341956 A1
(43) Pub. Date: Nov. 20, 2014

(54) ANTI-UV COMPOSITION FOR PROTECTING SCALP

(71) Applicants: Ki Ho Bang, Seoul (KR); LA CROIX CO., LTD., Seoul (KR)

(72) Inventors: Ki Ho BANG, Seoul (KR); Doo Kyung MOON, Seoul (KR); Dong Sung SEO, Daejeon (KR)

(73) Assignees: LA CROIX CO., LTD., Seoul (KR); Ki Ho Bang, Seoul (KR)

(21) Appl. No.: 14/280,960

(22) Filed: May 19, 2014

(30) Foreign Application Priority Data
May 20, 2013 (KR) 10-2013-0056440

(51) Int. Cl. A61K 8/44; A61K 8/97; A61K 8/37; A61Q 7/00; A61K 8/29; A61K 8/34; A61K 8/58; A61K 8/60; A61Q 17/04; A61K 8/35

(52) U.S. Cl. CPC A61K 8/445; A61Q 17/04; A61K 8/97; A61K 8/37; A61K 8/35; A61K 8/29; A61K 8/34; A61K 8/345; A61K 8/585; A61K 8/60; A61Q 7/00; A61K 2800/70
USPC 424/401; 424/60

(57) ABSTRACT

Disclosed herein is a anti-UV composition for protecting the scalp. The anti-UV composition for protecting the scalp of the present invention includes a fermented broth of Outtuynia cordata, Perilla frutescens, and Camellia sinensis; a UV blocking agent; and a pigment in an effective amount, respectively, thereby enabling the blocking of UV rays via scattering, reflecting, and absorbing, thus preventing scalp damage and alopecia due to UV while helping to recover scalp health and thus promoting the expression of new hair.

### BACKGROUND OF THE INVENTION

1. Field of the Invention

The present invention relates generally to an anti-UV composition for protecting the scalp.

2. Description of the Related Art

Sunlight is electromagnetic radiation given off by the sun that can be classified into gamma rays, x-rays, ultraviolet (UV) rays, visible rays, infrared rays, and radio waves, in order from the shortest to the longest wavelength. In addition, UV rays can be subdivided into UVC (200-280 nm), UVB (280-320 nm), and UVA (320-400 nm) depending on the wavelength size from the shortest to the longest. Since fatally harmful radiations such as UVC, gamma rays, and x-rays are absorbed and scattered by the ozone layer, water vapor, dust, etc., via absorption and scattering before they reach the surface of the earth, only 49% of visible light, 45% of infrared rays, and 6% of UV rays can reach the surface of the earth, as measured based on the amount being irradiated. However, although the irradiation amounts of UVA and UVB are much smaller than those of visible rays and infrared rays, people should pay more heed to them to prevent exposure thereto because of their extreme harmfulness to human bodies.

UVA has a relatively weak energy due to its long wavelength and can pass through glass, penetrate deep into the reticular layer of the dermis, and convert light-colored melanin pigments present in the skin surface into dark-colored ones, thus darkening the skin. Besides, UVA can modify collagen and elastin in the skin tissues and deteriorate their elasticity thereby stimulating skin aging and also causing photo-sensitive skin diseases. Because UVA can directly reach the surface of the earth even on cloudy days by passing through fog or windows, people should always protect themselves from possible exposure to UVA during their daily lives.

UVB is a high energy radiation with the shortest wavelength among the solar radiations that arrive at the surface of the earth. UVB can promptly penetrate the skin surface and the upper layer of the dermis of humans, thereby causing skin erythema, sunburns, facial flushing, blisters, and rashes. A few days after irradiation, UVB causes an increase in melanin to cause pigmentation on the skin.

Upon irradiation on facial skin, UVB can penetrate into the lower layer of the dermis unlike UVA, which can only reach the upper layer of dermis. Because the scalp is thinner than facial skin, UVB with a greater energy can penetrate into the upper layer of dermis while UVA can completely penetrate into the lower layer of dermis. Therefore, the scalp becomes more vulnerable to UV damage than facial and body skin. Nevertheless, many people do not take efforts to protect their scalp, the human body part closest to and most readily exposed to sunlight, from UV rays.

As the related arts, Korean Patent Application Publication No. 2008-0020857 discloses shampoo compositions containing microcapsules filled with anti-UV ingredients and fragrances, and Korean Patent No. 0774973 discloses a hair care composition. However, there has been no report of a technology relating to an anti-UV composition for protecting the scalp capable of preventing damage on scalp thereby enabling the prevention of hair loss while promoting hair growth.

### SUMMARY OF THE INVENTION

Accordingly, the present invention has been made keeping in mind the above problems occurring in the prior art, and an object of the present invention is to provide a Anti-UV composition for protecting the scalp capable of preventing scalp damage and hair loss due to UV and also a rejuvenating scalp thereby enabling hair growth.

In an effort to achieve the above object, the present invention provides an anti-UV composition for protecting the scalp containing at least one UV blocking agent selected from the group consisting of organic blocking agents and inorganic blocking agents; and at least one pigment selected from the group consisting of a spherical pigment with a particle size from 50 to 500 nm, a flake pigment with a particle size from 0.5 to 20 μm, a titanium dioxide/dopant sinter pigment and a zinc oxide/dopant sinter pigment; as active ingredients, wherein the dopant is a metal oxide or titanium.

Examples of the organic UV blocking agent may include glyceryl PABA; drometrizole, digaloyl trioleate; 3,(4-methylbenzylidene)camphor; methyl anthranilate; benzophenone-3; benzophenone-4; benzophenone-8; butyl methoxydibenzoylmethane; cinoxate; octocrylene; ethylhexyl dimethyl PABA; ethylhexyl methoxycinnamate; ethylhexyl salicylate; ethylhexyl triazone; p-aminobenzoic acid (PABA); 2-phenylbenzimidazole-5-sulfonic acid; homosalate; isoamyl-p-methoxycinnamate; bis-ethylhexyloxyphenol methoxyphenyl triazone; disodium phenyl dibenzimidazole tetrasulfonate; drometrizole trisiloxane; diethylhexyl butamido triazone (Diethylhexyl butamido triazone); polysilicone-15 or dimethicodiethyl benzal malonate; methylene bis-benzotriazolyl tetramethylbutylphenol; terephthalylidene dicamphor sulfonic acid and diethylamino hydroxybenzoyl hexyl benzoate.

As the organic UV blocking agent, from glyceryl PABA may be used in an amount of from 0.25 to 3.0 wt %, drometrizole in an amount of from 0.25 to 7.0 wt %, digaloyl trioleate in an amount of from 0.25 to 5.0 wt %, 3,(4-methylbenzylidene)camphor in an amount of from 0.25 to 5.0 wt %, methyl anthranilate in an amount of from 0.25 to 5.0 wt %, benzophenone-3 in an amount of from 0.25 to 5.0 wt, benzophenone-4 in an amount of from 0.25 to 5.0 wt %, benzophenone-8 in an amount of from 0.25 to 3.0 wt %, butyl methoxydibenzoylmethane in an amount of from 0.25 to 5.0 wt %, cinoxate in an amount of from 0.25 to 5.0 wt %, octocrylene in an amount of from 0.25 to 10.0 wt %, ethylhexyl dimethyl PABA in an amount of from 0.25 to 8.0 wt %, ethylhexyl methoxycinnamate in an amount of from 0.25 to 7.0 wt %, ethylhexyl salicylate in an amount of from 0.25 to 5.0 wt %, ethylhexyl triazone in an amount of from 0.25 to 5.0 wt %, PABA in an amount of from 0.25 to 5.0 wt %, 2-phenylbenzimidazole-5-sulfonic acid in an amount of from 0.25 to 4.0 wt %, homosalate in an amount of from 0.25 to 10.0 wt %, isoamyl-p-methoxycinnamate in an amount of from 0.25 to 10.0 wt %, bis-ethylhexyloxyphenol methoxyphenyl triazone in an amount of from 0.25 to 10.0 wt %, disodium phenyl dibenzimidazole tetrasulfonate in an amount of from 0.25 to 10.0 wt %, drometrizole trisiloxane in an amount of from 0.25 to 15.0 wt %, diethylhexyl butamido triazone in an amount of from 0.25 to 10.0 wt %, polysilicone-15 in an amount of from 0.25 to 10.0 wt %, methylene bis-benzotriazolyl tetramethylbutylphenol in an amount of from 0.25 to 10.0 wt %, terephthalylidene dicamphor sulfonic acid in an amount of from 0.25 to 10.0 wt %, or diethylamino hydroxybenzoyl hexyl benzoate in an amount of from 0.25 to 10.0 wt %.

The inorganic UV blocking agent may be titanium dioxide or zinc oxide.

In this regard, titanium dioxide and zinc oxide may be used in an amount of from 0.1 to 12.5 wt %, and from 0.1 to 12.5 wt %, respectively.

The pigment with a particle size from 50 to 500 nm may be barium sulfate (BaSO₄), spherical silica (SiO₂) or alumina (Al₂O₃).

The pigment with a particle size from 0.5 to 20 μm may be talc, mica, sericite, biotite, boron nitride, guanine, N ε-Lauroyl-L-lysine, bismuth oxychloride or titanium dioxide coated mica.

The pigment of the titanium dioxide/dopant sinter may be selected from the group consisting of a titanium dioxide/titanium sinter, a titanium dioxide/yellow iron oxide sinter, a titanium dioxide/red iron oxide sinter, a titanium dioxide/black iron oxide sinter, a titanium dioxide/yellow iron oxide/red iron oxide sinter, a titanium dioxide/yellow iron oxide/black iron oxide sinter, and a titanium dioxide/yellow iron oxide/red iron oxide/black iron oxide sinter.

More specifically, the pigment of the titanium dioxide/dopant sinter may include any one selected from the group in the amount ranging from 0.5 to 12.5 wt % for the titanium dioxide/titanium sinter, from 0.5 to 10.0 wt % for the titanium dioxide/yellow iron oxide sinter, from 0.5 to 10.0 wt % for the titanium dioxide/red iron oxide sinter, from 0.5 to 5.0 wt % for the titanium dioxide/black iron oxide sinter, from 0.5 to 10.0 wt % for the titanium dioxide/yellow iron oxide/red iron oxide sinter, from 0.5 to 5.0 wt % for the titanium dioxide/yellow iron oxide/black iron oxide sinter, and from 0.5 to 5.0 wt % for the titanium dioxide/yellow iron oxide/red iron oxide/black iron oxide sinter.

The pigment of the zinc oxide/dopant sinter may be selected from the group consisting of a zinc oxide/yellow iron oxide sinter, a zinc oxide/red iron oxide sinter, a zinc oxide/black iron oxide sinter, a zinc oxide/yellow iron oxide/red iron oxide sinter, a zinc oxide/yellow iron oxide/black iron oxide sinter and a zinc oxide/yellow iron oxide/red iron oxide/black iron oxide sinter.

More specifically, the pigment of the zinc oxide/dopant sinter may include any one selected from the group in the amount ranging from 0.5 to 10.0 wt % for the zinc oxide/yellow iron oxide sinter, from 0.5 to 10.0 wt % for the zinc oxide/red iron oxide sinter, from 0.5 to 5.0 wt % for the zinc oxide/black iron oxide sinter, from 0.5 to 10.0 wt % for the zinc oxide/yellow iron oxide/red iron oxide sinter, from 0.5 to 5.0 wt % for the zinc oxide/yellow iron oxide/black iron oxide sinter, and from 0.5 to 5.0 wt % for the zinc oxide/yellow iron oxide/red iron oxide/black iron oxide sinter.

The present invention also provides an anti-UV composition for protecting the scalp including a fermented broth prepared by fermentation of a mixture containing Houttuynia cordata, Perilla frutescens and Camellia sinensis; at least one UV blocking agent selected from the group consisting of organic UV blocking agents and inorganic UV blocking agents; and a pigment selected from the group consisting of a spherical pigment with a particle size from 50 to 500 nm, a flake pigment with a particle size from 0.5 to 20 μm, a titanium dioxide/dopant sinter pigment and a zinc oxide/dopant sinter pigment as active ingredients, wherein the dopant is a metal oxide or titanium.

The fermented broth may be manufactured by a method which includes (a) adding a fermentation agent to the mixture comprising Houttuynia cordata, Perilla frutescens and Camellia sinensis and leaving the resultant as it is; (b) adding ethanol to the resultant after the fermentation process in step (a); (c) performing fermentation and curing at from 15 to 25° C.; and (d) filtering the resultant obtained in step (c).

In particular, in step (a) above, the fermentation agent is nuruk or yeast.

The fermented broth is added from 0.1 to 30.0 wt %.

As described hitherto, the Anti-UV composition for protecting the scalp of the present invention includes a fermented broth mixture of Outtuynia cordata, Perilla frutescens, and Camellia sinensis; a UV blocking agent; and a pigment in an effective amount, respectively, thereby enabling the blocking of UV rays via scattering, reflecting, and absorbing, thus preventing scalp damage and alopecia due to UV while helping to recover scalp health and thus promoting expression of new hair.

Additionally, the Anti-UV composition for protecting the scalp of the present invention prevents of a decrease in the thickness of scalp, the degeneration and decrease of blood flow of capillaries in the dermis, and also the decrease in elasticity and flexibility of collagen fibers and elastin fibers, and is thus capable of facilitating a healthy scalp.

### BRIEF DESCRIPTION OF THE DRAWINGS

The above and other objects, features and other advantages of the present invention will be more clearly understood from the following detailed description taken in conjunction with the accompanying drawings, in which:

FIG. 1 shows a picture of a subject having hair loss on the top (A) of the head but no hair loss on the side (B) of the head;

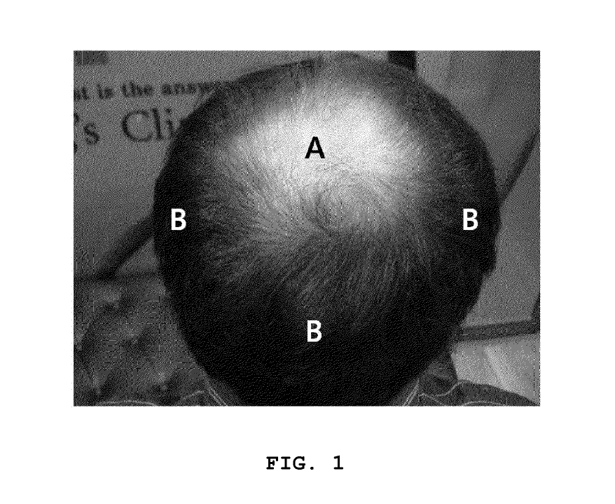

FIG. 2 shows a picture of a subject having an UV damaged scalp region on the top (A) of the head, and the normal scalp region on the side (B) of the head, as taken with a digital microscope (Aramo-SG Diagnosis System, Aramhuvis Co., Ltd., USA);

FIG. 3 shows an ultrasonic image showing the tissue state and thickness of the scalp region on the top (A) of the head, and the side (B) of the head obtained by an ultrasonic tomography system;

FIG. 4 is a graph showing the UV transmittance of a fermented broth mixture of Houttuynia cordata, Perilla frutescens and Camellia sinensis prepared in Example 1 of the present invention by UV-VIS Spectrophotometer at the wavelength of 200-400 nm;

FIG. 5 is a digital microscopic view of the scalp vertex after application of the Anti-UV composition according to the present invention to the scalp, showing the appearance of new hair strands from the scalp; and

FIG. 6 shows the scalp before and after application of the Anti-UV composition according to the present invention to the scalp, demonstrating the effect of the composition on the prevention of hair loss.

### DESCRIPTION OF THE PREFERRED EMBODIMENTS

Reference now should be made to the drawings, in which the same reference numerals are used throughout the different drawings to designate the same or similar components.

The present invention is described in greater detail infra.

The term scalp, used herein, refers to a skin covering the top of the human head including all parts of the head where hairs were present prior to hair loss exclusive of the face and neck.

The present invention also provides an Anti-UV composition for the scalp including at least one UV blocking agent selected from the group consisting of organic UV blocking agents and inorganic UV blocking agents; and a pigment selected from the group consisting of a spherical pigment with a particle size from 50 to 500 nm, a platy pigment with a particle size from 0.5 to 20 μm, a titanium dioxide/dopant sinter pigment, and a zinc oxide/dopant sinter pigment; as active ingredients, wherein the dopant is a metal oxide or titanium.

Preferably, the organic UV blocking agent may be selected from the group consisting of glyceryl PABA, drometrizole, digaloyl trioleate, 3,(4-methylbenzylidene)camphor, methyl anthranilate, benzophenone-3, benzophenone-4, benzophenone-8, butyl methoxydibenzoylmethane, cinoxate, octocrylene, ethylhexyl dimethyl PABA, ethylhexyl methoxycinnamate, ethylhexyl salicylate, ethylhexyl triazone, p-aminobenzoic acid (PABA), 2-phenylbenzimidazole-5-sulfonic acid, homosalate, isoamyl-p-methoxycinnamate, bis-ethylhexyloxyphenol methoxyphenyl triazone, disodium phenyl dibenzimidazole tetrasulfonate, drometrizole trisiloxane, diethylhexyl butamido triazone (Diethylhexyl butamido triazone), polysilicone-15 or dimethicodiethyl benzal malonate, methylene bis-benzotriazolyl tetramethylbutylphenol, terephthalylidene dicamphor sulfonic acid and diethylamino hydroxybenzoyl hexyl benzoate.

More specifically, the organic UV blocking agent may exhibit an optimal effect when it includes any one selected from the group in the amount ranging from 0.25 to 3.0 wt % of glyceryl PABA, from 0.25 to 7.0 wt % of drometrizole, from 0.25 to 5.0 wt % of digaloyl trioleate, from 0.25 to 5.0 wt % of 3,(4-methylbenzylidene)camphor, from 0.25 to 5.0 wt % of methyl anthranilate, from 0.25 to 5.0 wt % of benzophenone-3, from 0.25 to 5.0 wt % of benzophenone-4, from 0.25 to 3.0 wt % of benzophenone-8, from 0.25 to 5.0 wt % of butyl methoxydibenzoylmethane, from 0.25 to 5.0 wt % of cinoxate, from 0.25 to 10.0 wt % of octocrylene, from 0.25 to 8.0 wt % of ethylhexyl dimethyl PABA, from 0.25 to 7.0 wt % of ethylhexyl methoxycinnamate, from 0.25 to 5.0 wt % of ethylhexyl salicylate, from 0.25 to 5.0 wt % of ethylhexyl triazone, from 0.25 to 5.0 wt % of PABA, from 0.25 to 4.0 wt % of 2-phenylbenzimidazole-5-sulfonic acid, from 0.25 to 10.0 wt % of homosalate, from 0.25 to 10.0 wt % of isoamyl-p-methoxycinnamate, from 0.25 to 10.0 wt % of bis-ethylhexyloxyphenol methoxyphenyl triazone, from 0.25 to 10.0 wt % of disodium phenyl dibenzimidazole tetrasulfonate, from 0.25 to 15.0 wt % of drometrizole trisiloxane, from 0.25 to 10.0 wt % of diethylhexyl butamido triazone, from 0.25 to 10.0 wt % of polysilicone-15, from 0.25 to 10.0 wt % of methylene bis-benzotriazolyl tetramethylbutylphenol, from 0.25 to 10.0 wt % of terephthalylidene dicamphor sulfonic acid, and from 0.25 to 10.0 wt % of diethylamino hydroxybenzoyl hexyl benzoate.

Preferably, the inorganic UV blocking agent is titanium dioxide or zinc oxide.

Preferably, titanium dioxide is added from 0.1 to 12.5 wt %, and zinc oxide is added from 0.1 to 12.5 wt %.

As described above, the UV blocking agent, even in a lesser amount, can exhibit a more efficient UV blocking effect than that of the conventional art. This is because it can scatter and reflect UV rays due to the pigments contained therein as described below. The pigments of the present invention enable efficient blocking of UV rays thereby preventing adverse effects and anaphylaxis of skin.

In general, pigments reveal their own colors by reflecting or scattering wavelengths in a particular range of the visible spectra upon irradiation by sunlight. However, in a region where the wavelength of sunlight is shorter than that of a visible ray, the wavelength selectively penetrates the pigment depending on the conditions, thus delimiting the use of the pigments as a method for blocking UV rays. In light of optical phenomena, the scattering, reflection, and penetration of irradiated rays appear to depend on the size, shape, and refractive index of pigments. In addition, the absorption of rays is associated with the intrinsic band gap energy of a pigment, and UV blocking capability varies depending on whether the blocking utilizes any combination of scattering, reflection, penetration, absorption, etc.

Preferably, the pigment of the present invention is added from 1.0 to 20.0 wt %.

Preferably, the pigments with a particle size from 50 to 500 nm are barium sulfate (BaSO₄), spherical silica (SiO₂) or alumina (Al₂O₃). Pigments may exhibit the optimal effect when added from 0.1 to 12.5 wt % to the Anti-UV composition. Pigments may optimize the ray scattering effect when the particle diameter of a given pigment is about a half of the wavelength of the ray being irradiated thereon. Therefore, in order to scatter UV rays in the range of 200 to 400 nm, the particle diameter of the pigments is preferably in the range of 100 to 200 nm. In this regard, the particle diameter of the pigments of the present invention to be used as a component for the Anti-UV composition for protecting the scalp is preferably in the range of from 50 to 500 nm, considering the distribution state of the pigments in a medium.

Preferably, the pigments with a particle size from 0.5 to 20 μm are talc, mica, sericite, biotite, boron nitride, guanine, N ε-Lauroyl-L-lysine, bismuth oxychloride or titanium dioxide coated mica. Preferably, the pigments that reflect rays have a platy shape, and have a particle diameter larger than the wavelength of the ray to be irradiated thereon. When the pigments are applied on the scalp they serve as a mirror plane to reflect UV rays. In particular, the higher the whiteness of a given pigment the greater the reflecting efficiency, and mica coated with an iron oxide can exhibit the optimum effect.

Preferably, the pigment of the titanium dioxide/dopant sinter may be selected from the group consisting of a titanium dioxide/titanium sinter, a titanium dioxide/yellow iron oxide sinter, a titanium dioxide/red iron oxide sinter, a titanium dioxide/black iron oxide sinter, a titanium dioxide/yellow iron oxide/red iron oxide sinter, a titanium dioxide/yellow iron oxide/black iron oxide sinter and a titanium dioxide/yellow iron oxide/red iron oxide/black iron oxide sinter.

Preferably, in order to exhibit the optimum effect, the titanium dioxide/titanium sinter is mixed from 0.5 to 12.5 wt %, the titanium dioxide/yellow iron oxide sinter from 0.5 to 10.0 wt %, the titanium dioxide/red iron oxide sinter from 0.5 to 10.0 wt %, the titanium dioxide/black iron oxide sinter from 0.5 to 5.0 wt %, the titanium dioxide/yellow iron oxide/red iron oxide sinter from 0.5 to 10.0 wt %, the titanium dioxide/yellow iron oxide/black iron oxide sinter from 0.5 to 5.0 wt % of, and the titanium dioxide/yellow iron oxide/red iron oxide/black iron oxide sinter from 0.5 to 5.0 wt %, respectively.

Preferably, the titanium dioxide/titanium sinter may be mixed in the weight ratio from 95:5 to 80:20, the titanium dioxide/yellow iron oxide sinter from 95:5 to 30:70, the titanium dioxide/red iron oxide sinter from 98:2 to 50:50, the titanium dioxide/black iron oxide sinter from 98:2 to 70:30, the titanium dioxide/yellow iron oxide/red iron oxide sinter from 93:5:2 to 40:30:30, the titanium dioxide/yellow iron oxide/black iron oxide sinter from 93:5:2 to 40:30:30, the titanium dioxide/yellow iron oxide/red iron oxide/black iron oxide sinter from 91:5:2:2 to 40:20:20:20, respectively, and sintered thereafter.

Preferably, the pigment of the zinc oxide/dopant sinter may be selected from the group consisting of a zinc oxide/yellow iron oxide sinter, a zinc oxide/red iron oxide sinter, a zinc oxide/black iron oxide sinter, a zinc oxide/yellow iron oxide/red iron oxide sinter, a zinc oxide/yellow iron oxide/black iron oxide sinter or a zinc oxide/yellow iron oxide/red iron oxide/black iron oxide sinter.

Preferably, in order to exhibit the optimum effect, the zinc oxide/yellow iron oxide sinter is mixed from 0.5 to 10.0 wt %, the zinc oxide/red iron oxide sinter from 0.5 to 10.0 wt %, the zinc oxide/black iron oxide sinter from 0.5 to 5.0 wt %, the zinc oxide/yellow iron oxide/red iron oxide sinter from 0.5 to 10.0 wt %, the zinc oxide/yellow iron oxide/black iron oxide sinter from 0.5 to 5.0 wt %, and the zinc oxide/yellow iron oxide/red iron oxide/black iron oxide sinter from 0.5 to 5.0 wt %, respectively.

Preferably, the zinc oxide/yellow iron oxide sinter may be mixed in the weight ratio from 95:5 to 30:70, the zinc oxide/red iron oxide sinter from 98:2 to 50:50, the zinc oxide/black iron oxide sinter from 98:2 to 70:30, the zinc oxide/yellow iron oxide/red iron oxide sinter from 93:5:2 to 40:30:30, the zinc oxide/yellow iron oxide/black iron oxide sinter from 93:5:2 to 40:30:30, the zinc oxide/yellow iron oxide/red iron oxide/black iron oxide sinter from 91:5:2:2 to 40:20:20:20, and sintered thereafter.

Examples of the pigments to be used as a component for the Anti-UV composition for protecting the scalp of the present invention may include a yellow iron oxide/red iron oxide sinter pigment and a yellow iron oxide/red iron oxide/black iron oxide sinter pigment. Preferably, the yellow iron oxide/red iron oxide sinter pigment should be mixed from 0.1 to 3.0 wt %, and the yellow iron oxide/red iron oxide/black iron oxide sinter pigment should be mixed from 0.1 to 3.0 wt %. In addition, the yellow iron oxide/red iron oxide sinter is preferably mixed in a weight ratio from 90:10 to 50:50, and the yellow iron oxide/red iron oxide/black iron oxide sinter from 90:5:5 to 60:20:20.

A metal oxide pigment takes on a semi-conductive property upon light irradiation thereon, and it absorbs the ray with a wavelength corresponding to the band gap energy between a valence band of the pigment and the conductive band. The band gap energy corresponding to UV in the range of 200 to 400 nm is 6.2˜3.1 eV, and the band gap energy corresponding to UVA, which can arrive at the surface of the earth in the range of 320 to 400 nm, is 3.8˜3.1 eV.

Due to the high refractive index, titanium dioxide and zinc oxide can effectively refract and scatter rays thus effectively blocking UV rays. Furthermore, titanium dioxide and zinc oxide have a band gap energy of 3.2 eV and enable the absorption of a UV wavelength of 388 nm corresponding to UVA. In addition, when they are doped with iron oxide or titan metal with a band gap energy of 2.3 eV, their absorption wavelengths can be shifted to absorb the wavelengths in the range of 388 to 539 nm, and are thus capable of absorbing UVA and the rays that correspond to the blue wavelength region of the visible rays that can penetrate into the scalp. Besides, the dopants such as a metal oxide and titanium of the present invention can help a sinter to take on a color. Accordingly, by doping the white-colored titanium dioxide and zinc oxide with a dopant, their colors can be variously changed into a pale yellow similar to the color of scalp; beige, a color similar to hair color; blue with a glowing black; brown, similar to the skin color of black people; or brown black, etc., thereby solving the problems of the white-colored Anti-UV composition having an imbalance with scalp and hairs.

The sinter pigments may be manufactured in a sintering furnace under an oxidizing or reducing atmosphere at 600 to 950° C. from 1 hour to 8 hours. Additionally, the present invention also provides a Anti-UV composition for protecting the scalp including a fermented broth prepared by fermentation of a mixture containing Houttuynia cordata, Perilla frutescens and Camellia sinensis; at least one UV blocking agent selected from the group consisting of organic UV blocking agents and inorganic UV blocking agents; and a pigment selected from the group consisting of a pigment with a particle size from 50 to 500 nm, a pigment with a particle size from 0.5 to 20 μm, a titanium dioxide/dopant sinter pigment, and a zinc oxide/dopant sinter pigment as active ingredients, wherein the dopant is a metal oxide or titanium.

The fermented broth may be manufactured by a method which includes (a) adding a fermentation agent to the mixture comprising Houttuynia cordata, Perilla frutescens and Camellia sinensis, and leaving the resultant as it is; (b) adding ethanol to the resultant after the fermentation process in step (a); (c) performing fermentation and curing at from 15 to 25° C. with 50 to 60% humidity; and (d) filtering the resultant obtained in step (c). Preferably, Houttuynia cordata, Perilla frutescens and Camellia sinensis are mixed in a weight ratio ranging from 1 to 8:1 to 8:1 to 8.

In step (a), the fermentation agent, either nuruk or yeast, is preferably added at 1/50 of the total weight of the mixture containing Houttuynia cordata, Perilla frutescens and Camellia sinensis. The fermented broth manufactured by the method exhibited the optimal effect when it was mixed from 0.1 to 30.0 wt %, preferably from 0.5 to 20.0 wt %, relative to the Anti-UV composition the for protecting scalp.

Houttuynia cordata is a perennial plant belonging to family Saururaceae and has strong antibacterial and antiinflammatory effects. More specifically, decanoyl acetaldehyde present in Houttuynia cordata has an excellent inhibitory effect against Malassezia yeast and Staphylococcus sp., which are known to cause and aggravate seborrheic alopecia.

Perilla frutescens is an annual plant belonging to Labiata family, and is also called as Perilla frutescens var. It contains a large amount of minerals, vitamins B1, B2, B6, C, E, K, niacin, and other elements such as alpha-linoleic acid and is involved in the decomposition of body cholesterol, calcium, potassium, iron, magnesium, zinc, etc., thus it virtually contains all essential coenzyme components. Perilla frutescens also contains perillaldehyde and beta-carotene, thus has strong antibacterial and antioxidizing effects. Furthermore, phytochemicals present in Perilla frutescens help to activate hair follicle cells while at the same time prevent the aging process. Accordingly, Perilla frutescens in combination with Houttuynia cordata exhibits an optimal synergistic effect to alleviate seborrheic and male pattern alopecia.

Camellia sinensis contains various components including polyphenol, caffeine, amino acids, etc. Therefore, Camellia sinensis, along with Houttuynia cordata and Perilla frutescens, establishes a triple effect of antioxidation, antiinflammation, and anti-DHT, thus improving the intended effect of the Anti-UV composition for protecting the scalp of the present invention.

The Anti-UV composition for protecting the scalp of the present invention may be applied to cosmetics such as shampoos, rinses, hair treatments, hair tonics, hair lotions, and hair oils. Furthermore, it may be also added to pharmaceutical products such as ointments, creams, patches, semi-transparent non-woven fabrics, etc.

The following non-limiting Examples serve to illustrate exemplary embodiments of the invention. It will be appreciated that variations in proportions and alternatives in elements of the components shown will be apparent to those skilled in the art and are within the scope of embodiments of the present invention.

### Example 1

A 10 L ripening container for fermentation was sterilized and then added with trimmed raw herbal materials for fermentation as follows.

Fresh non-dried leaves of Houttuynia cordata were trimmed, cut into a size with 1 cm of width in a landscape direction, and then held until a transparent viscous liquid came out of the sections when pressed with a finger. Upon confirmation of the above, the cut-out leaves were put into the container for fermentation. Meanwhile, Perilla frutescens and Camellia sinensis were used in a dry state and the leaves were cut into an appropriate size.

Houttuynia cordata, Perilla frutescens and Camellia sinensis were added to the fermentation container in the amount of 250 g, 150 g, and 100 g, respectively, to a total of 500 g, in a weight ratio of 50:30:20. Then, 10 g of nuruk was further added thereto, well mixed with the above three raw herbal materials, and sealed with a lid. After leaving the resultant for 2 days as it was, the lid of the fermentation container was opened and 8.0 L of 30% ethanol was carefully decanted. The fermentation container was slowly shaken to mix the contents therein and sealed again with a lid.

The materials described above were sealed in a fermentation container and were fermented and cured in an indoor place shielded from visible rays, and kept at about 20° C. with about 55% of humidity for 6 months. During the fermentation, the sealed fermentation container was rolled about once a week for 10 minutes to sufficiently mix the nuruk, raw herbal materials and ethanol.

After 6 months, the lid of the fermentation container was opened, and a fermented broth was obtained by separating a liquid from the residue of the raw herbal materials. The thus obtained fermented broth was filtrated and 7.5 L of a fermented broth of Houttuynia cordata, Perilla frutescens, and Camellia sinensis was finally obtained.

### Example 2

To a ball mill were added 70 g of titanium dioxide (TiO₂) and 25 g of yellow iron oxide (FeOOH) along with 300 mL of purified water, and then treated for 4 hours therein. The ball mill-treated pulverized titanium dioxide-yellow iron oxide was transferred into a 1000 mL beaker, and slowly stirred with a mixer. Then, a solution, where 15 g of ferric sulfate (Fe₂(SO₄)₃) was dissolved in 300 mL of purified water, being used as a precursor for the doping of red iron oxide (Fe₂O₃), was added thereto, and 10% ammonia water was slowly added dropwise to the beaker to initiate a reaction with the contents being stirred therein. If the viscosity of the pulverized in the beaker rapidly increased, 1 or 2 drops of 10% ammonia water was added to the beaker, and stirring was terminated after 10 minutes. Then, the resultant was washed twice with purified water, filtrated, dried and then pulverized into powder by a pulverizer.

The thus obtained powder was put into an alumina crucible and sintered in an electric furnace kept at 820° C. for 2 hours, and finally 101 g of skin-colored beige titanium dioxide/yellow iron oxide/red iron oxide sinter was obtained.

### Examples 3-6

A Anti-UV composition for protecting the scalp including the fermented broth and the UV blocking agent prepared in Example 1, and the pigment of the titanium dioxide/yellow iron oxide/red iron oxide sinter prepared in Example 2 was manufactured as shown in Table 1 below.

More specifically, the components of a pigment region were dispersed while heating at 80° C. after adding them into a water phase region, and concurrently, the UV blocking agent was added into the oil phase region and dissolved by heating at 80° C. The heat dissolved oil phase region was sufficiently emulsified by adding it into the water phase region, and slowly cooled to room temperature while stirring the emulsion in a water bath, thereby obtaining products corresponding to each of the Examples.

TABLE 1

| Category | Component (wt %) | Example 3 | Example 4 | Example 5 | Example 6 |
|---|---|---|---|---|---|
| Aqueous | 1. Purified water | To 100 | To 100 | To 100 | To 100 |
| phase | 2. Ethanol | 5.0 | 5.0 | 5.0 | 5.0 |
| region | 3. 1,3-butylene | 5.0 | 5.0 | 5.0 | 5.0 |
| | glycol | | | | |
| | 4. Sodium | 0.5 | 0.5 | 0.5 | 0.5 |
| | hyarulonate | | | | |
| | 5. Fermented broth | 10.0 | 5.0 | 5.0 | 5.0 |
| | prepared in | | | | |
| | Example 1 | | | | |
| Oil | 6. Cyclomethicone | 7.0 | 7.0 | 7.0 | 7.0 |
| phase | 7. Dimethicone/vinyl | 3.5 | 3.5 | 3.5 | 3.5 |
| region | dimethicone | | | | |
| | crosspolymer | | | | |
| | 8. Caprylic/Capric | 3.0 | 3.0 | 3.0 | 3.0 |
| | triglyceride | | | | |
| | 9. PEG-10 | 1.0 | 1.0 | 1.0 | 1.0 |
| | dimethicone | | | | |
| | 10. Quaternium-18 | 0.5 | 0.5 | 0.5 | 0.5 |
| | hectorite | | | | |
| | 11. Preservative | Adequate | Adequate | Adequate | Adequate |
| | 12. Flavor | Adequate | Adequate | Adequate | Adequate |
| UV | 13. Glyceryl PABA | — | 3.0 | — | 1.5 |
| blocking | 14. Ethylhexyl | 3.0 | — | — | 1.5 |
| agent | methoxycinnamate | | | | |
| | 15. Butyl | — | — | 3.0 | — |
| | methoxydibenzoylmethane | | | | |
| Pigment | 16. Titanium | — | — | 5.0 | — |
| region | dioxide | | | | |
| | 17. Sinters | 5.0 | — | — | 10.0 |
| | prepared in | | | | |
| | Example 2 | | | | |

### Experimental Example 1

In order to study the effect of UV on the scalp, the scalps of a total of 200 alopecia patients and potential alopecia patients of were examined and their states were diagnosed as follows.

First, the patients having a distinct boundary between the region with hair loss and the normal region were singled out for clinical tests based on the observation of their scalp state by the naked eye. As shown in Table 1, he scalp state of the thus selected patients was divided into a top region (A) of the head with hair loss and a side or rear region (B) of the head not yet having hair loss, and observed under digital microscope (Aramo-SG Diagnosis System, Aramhuvis Co., Ltd., USA), respectively.

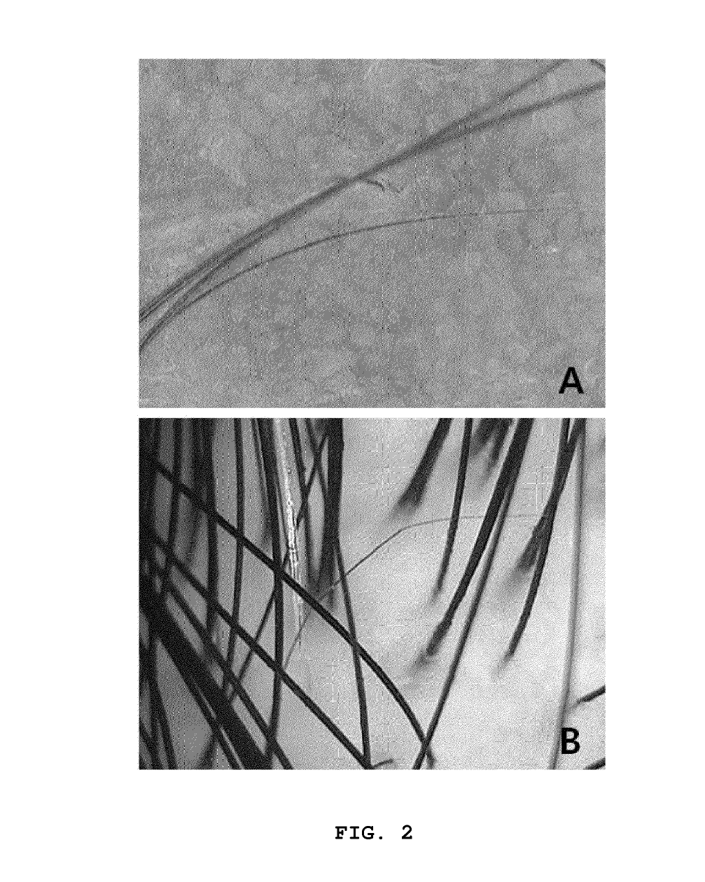

As shown in FIG. 2, the result revealed that the scalp on the top region (A) of the head with hair loss showed numerous grain-like shapes mimicking a honeycomb with red fine cracks being intermingled in a particular way thereon, whereas the side or rear region (B) of the head did not show anything similar to the honeycomb but retained the white and clear state.

Additionally, in order to study the level of scalp damage by UV irradiation, the inventors of the present invention developed an ultrasound system for scalp diagnosis, and examined the scalp state of subjects with normal scalps and those with damaged scalps. The ultrasound system developed by the present inventors can project an ultrasound signal with an intensity controlled to be suitable for healthy scalps, and it can produce images until the signal reaches deep into the scalp where then the signal is attenuated.

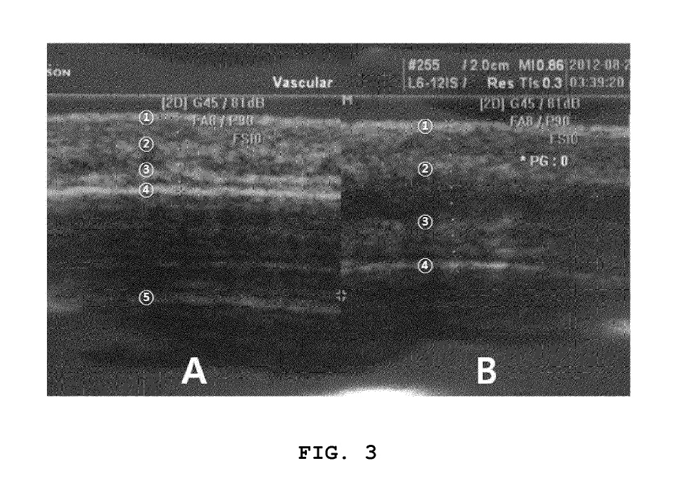

As shown in FIG. 3, the result revealed that the region (A) damaged by UV irradiation had a scalp thickness from 0.3-0.4 mm, whereas the normal region (B) had a scalp thickness from 0.6-0.7 mm, thus showing a 50% decrease in the thickness of the scalp damaged by UV irradiation. More specifically, the stratum corneum of the scalp \textcircled{1} showed almost no decrease in thickness but the thickness of the epidermis located below the stratum corneum between \textcircled{1} and \textcircled{2} showed a decrease, and in particular, a most substantial decrease was shown in the thickness of the dermis located between \textcircled{2} and \textcircled{3}. In addition, the blackish layer of collagen bundle annexed to the periosteum located in between the periosteum and the bone of the top of the head also showed a decrease in the thickness of the damaged scalp in the region (A). \textcircled{5} is a sort of an echo image produced by the ultrasound reflected from the bone of the top of the head. \textcircled{5} is a mirror image of the most external stratum corneum of the epidermis regenerated by the reflected ultrasound. Since the ultrasound projected to the thick normal scalp (B) traveled farther than that of the damaged scalp (A) it was deteriorated not to be reflected again as an echo from the dense bone of the top of the head. As a result, the mirror image of the stratum corneum was not observed on the normal scalp (B).

Accordingly, it was confirmed that UV rays penetrated into the papillary layer of the dermis can destroy the elasticity of collagen fibers and elastin fibers in the dermis, and shrivel capillaries, lymph ducts, neural fibers, hair follicle cells, and various secretory glands, etc., thereby causing transformation of scalp tissues.

### Experimental Example 2

The Anti-UV effect of the fermented broth of the mixture of Houttuynia cordata, Perilla frutescens, and Camellia sinensis prepared in Example 1 was measured at a wavelength of from 200 to 400 nm by UV-VIS spectrophotometer (HP Agilent 8453, USA).

Samples were measured using ethanol as a dilution solvent, and absolute ethanol as a blank sample. The UV transmittance of the fermented broth of the mixture of Houttuynia cordata, Perilla frutescens, and Camellia sinensis was measured and the results are shown in Table 4.

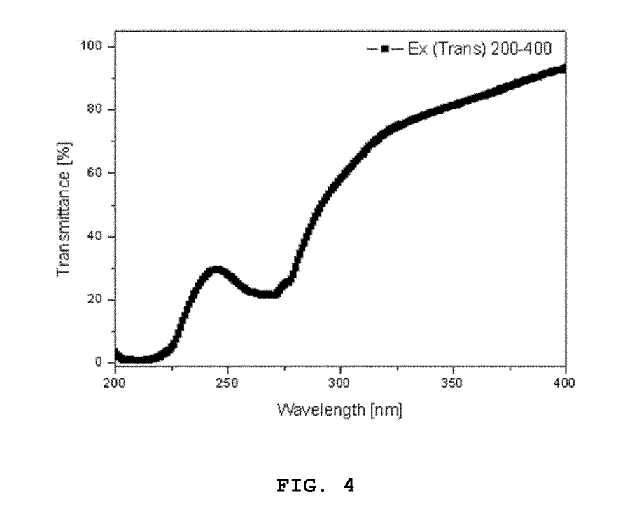

As shown in the spectrum of FIG. 4, the UV transmittance(%) measured at the 200 to 400 nm region was about 20% on average at the UVC (200-280 nm) region, about 50% on average at the UVB (280-320 nm) region, and about 80% on average at the UVA (320-400 nm region, respectively. Accordingly, it was found that the fermented broth of the mixture of Houttuynia cordata, Perilla frutescens, and Camellia sinensis has a UV ray protecting effect in UVC and UVB regions.

### Experimental Example 3

The Anti-UV composition prepared in Example 3 was added into brown glass bottles and provided to 50 outpatients who visited the hospital for the treatment of alopecia. The above outpatients were requested that for a period of 4 months they take a predetermined amount of the composition and apply it evenly on the entire area of the top of the head with hair loss in the morning before going out, and wash the area of the top of the head with in the evening after returning home. However, the information on the composition prepared in Example 3 was not given to the outpatients until completion of the evaluation but released thereafter.

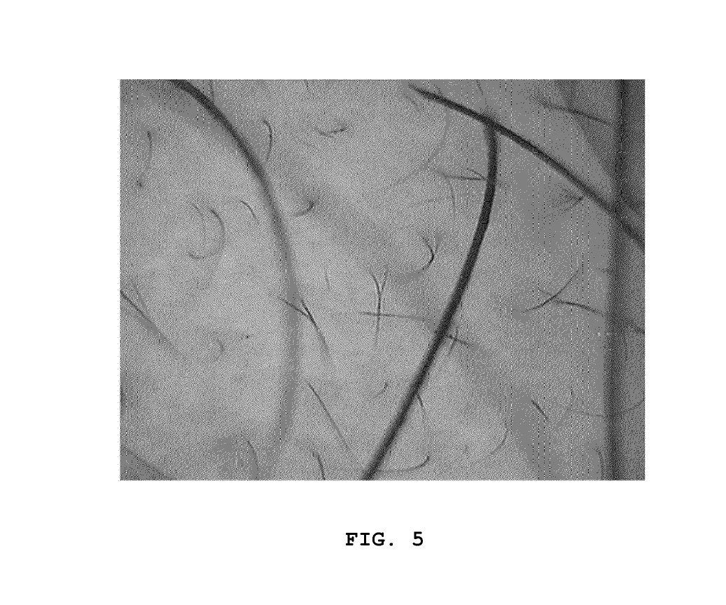

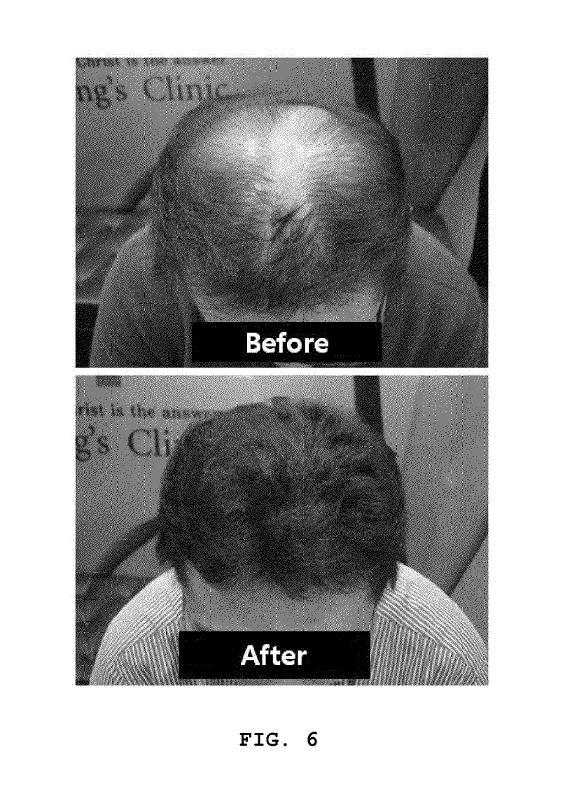

The pictures showing the observations of changes in scalp and improvement in hair loss and the results thereof are shown in FIGS. 5 and 6 and Table 2. The expression of new hair and the state of the scalp in the region with hair loss were observed and under a digital microscope and compared thereafter. The improvement in hair loss was evaluated by comparing the pictures taken before and after the application of the composition. The presence of any increase in the thickness of the damaged scalp was confirmed by the ultrasound system developed by the inventors of the present invention.

TABLE 2
Result of scalp after applying the Anti-UV composition
prepared in Example 3 (n = 50)

| | | | Little | Significant |
|---|---|---|---|---|
| Evaluation item | | No change | improvement | improvement |
| Scalp state | Increase in | — | 32 | 18 |
| | thickness | | | |
| | Presence of | — | 12 | 38 |
| | honeycomb | | | |
| | structure | | | |

| | | | |
|---|---|---|---|
| Expression of new hair | — | 14 | 36 |
| Improvement of scalp | — | 11 | 39 |
| inflammation | | | |
| Overall satisfaction | — | 8 | 42 |

In Table 2, evaluation was performed as follows: ‘no change’ when there was no or less than 10% of increase in thickness; ‘little improvement’ for 10 to 20% increase; and ‘significant improvement’ for 20% or higher of increase.

As a result, the clumpy red honeycomb-like feature on the UV damaged scalp disappeared, and there was an expression of new hair. Furthermore, upon evaluation by the ultrasound system, it was confirmed that the damaged scalp was recovering its thickness to the normal healthy level regardless of personal variations thereon. Furthermore, the examination by interview regarding the overall satisfaction revealed that the hair loss symptoms most patients experienced were alleviated or significantly improved, and scalp inflammation was reduced.

The Anti-UV compositions for scalp prepared in Examples 4 to exhibited the same effects. The Anti-UV composition for protecting the scalp of the present invention will be more effective for the scalp which is more prone to exposure to UV rays after the progress of hair loss.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. An anti-UV composition for protecting scalp comprising as active ingredients:
a UV blocking agent comprising 0.25 to 3.0 wt % of glyceryl PABA, 0.25 to 7.0 wt % of drometrizole; 0.25 to 5.0 wt % of digaloyl trioleate; 0.25 to 5.0 wt % of 3,(4-methylbenzylidene)camphor; 0.25 to 5.0 wt % of methyl anthranilate; 0.25 to 5.0 wt % of benzophenone-3; 0.25 to 5.0 wt % of benzophenone-4; 0.25 to 3.0 wt % of benzophenone-8; 0.25 to 5.0 wt % of butyl methoxydibenzoylmethane; 0.25 to 5.0 wt % of cinoxate; 0.25 to 10.0 wt % of octocrylene; 0.25 to 8.0 wt % of ethylhexyl dimethyl PABA; 0.25 to 7.0 wt % of ethylhexyl methoxycinnamate; 0.25 to 5.0 wt % of ethylhexyl salicylate; 0.25 to 5.0 wt % of ethylhexyl triazone; 0.25 to 5.0 wt % of PABA; 0.25 to 4.0 wt % of 2-phenylbenzimidazole-5-sulfonic acid; 0.25 to 10.0 wt % of homosalate; 0.25 to 10.0 wt % of isoamyl-p-methoxycinnamate; 0.25 to 10.0 wt % of bis-ethylhexyloxyphenol methoxyphenyl triazone; 0.25 to 10.0 wt % of disodium phenyl dibenzimidazole tetrasulfonate; 0.25 to 15.0 wt % of drometrizole trisiloxane; 0.25 to 10.0 wt % of diethylhexyl butamido triazone; 0.25 to 10.0 wt % of polysilicone-15; 0.25 to 10.0 wt % of methylene bis-benzotriazolyl tetramethylbutylphenol; 0.25 to 10.0 wt % of terephthalylidene dicamphor sulfonic acid; 0.25 to 10.0 wt % of diethylamino hydroxybenzoyl hexyl benzoate; and 0.1 to 12.5 wt % of titanium dioxide or 0.1 to 12.5 wt % of zinc oxide;
a pigment comprising 0.5 to 12.5 wt % of a sinter containing titanium dioxide and titanium; 0.5 to 10.0 wt % of a sinter containing titanium dioxide and yellow iron oxide; 0.5 to 10.0 wt % of a sinter containing titanium dioxide and red iron oxide; 0.5 to 5.0 wt % of a sinter containing titanium dioxide and black iron oxide; 0.5 to 10.0 wt % of a sinter containing titanium dioxide, yellow iron oxide and red iron oxide; 0.5 to 5.0 wt % of a sinter containing titanium dioxide, yellow iron oxide and black iron oxide; 0.5 to 5.0 wt % of a sinter containing titanium dioxide; yellow iron oxide, red iron oxide and black iron oxide; 0.5 to 10.0 wt % of a sinter containing zinc oxides and yellow iron oxide; 0.5 to 10.0 wt % of a sinter containing zinc oxides and red iron oxide; 0.5 to 5.0 wt % of a sinter containing zinc oxides and black iron oxide; 0.5 to 10.0 wt % of a sinter containing zinc oxides, yellow iron oxide and red iron oxide; 0.5 to 5.0 wt % of a sinter containing zinc oxides, yellow iron oxide and black iron oxide; 0.5 to 5.0 wt % of a sinter containing zinc oxides, yellow iron oxide, red iron oxide and black iron oxide; and 0.1 to 3.0 wt % of a sinter containing yellow iron oxide and red iron oxide or 0.1 to 3.0 wt % of a sinter containing yellow iron oxide, red iron oxide and black iron oxide; and
a fermented broth obtained by fermenting a mixture containing Houttuynia cordata, Perilla frutescens and Camellia sinensis,
wherein the pigment is sintered in a sintering furnace set at 600 to 950° C. under an oxidizing or reducing atmosphere for 1 to 8 hours.

2. The anti-UV composition of claim 1, wherein the pigment further comprises at least one selected from the group consisting of barium sulfate (BaSO₄), spherical silica (SiO₂), alumina (Al₂O₃), talc, mica, sericite, biotite, boron nitride, guanine, N ε-Lauroyl-L-lysine, bismuth oxychloride and titanium dioxide coated mica, wherein the particle size of barium sulfate (BaSO₄), spherical silica (SiO₂), and alumina (Al₂O₃) is from 50 to 500 nm, and the particle size of talc, mica, sericite, biotite, boron nitride, guanine, N ε-Lauroyl-L-lysine, bismuth oxychloride and titanium dioxide coated mica is from 0.5 to 20 μm.

3. The anti-UV composition of claim 1, wherein the pigment contains a sintered mixture of titanium dioxide and titanium at a weight ratio of from 95:5 to 80:20; titanium dioxide and yellow iron oxide at a weight ratio of from 95:5 to 30:70; titanium dioxide and red iron oxide at a weight ratio of from 98:2 to 50:50; titanium dioxide and black iron oxide at a weight ratio of from 98:2 to 70:30; titanium dioxide, yellow iron oxide and red iron oxide at a weight ratio of from 93:5:2 to 40:30:30; titanium dioxide, yellow iron oxide and black iron oxide at a weight ratio of from 93:5:2 to 40:30:30; titanium dioxide, yellow iron oxide, red iron oxide and black iron oxide at a weight ratio of from 91:5:2:2 to 40:20:20:20; zinc oxide and yellow iron oxide at a weight ratio of from 95:5 to 30:70; zinc oxide and red iron oxide at a weight ratio of from 98:2 to 50:50; zinc oxide and black iron oxide at a weight ratio of from 98:2 to 70:30; zinc oxide, yellow iron oxide and red iron oxide at a weight ratio of from 93:5:2 to 40:30:30; zinc oxide, yellow iron oxide and black iron oxide at a weight ratio of from 93:5:2 to 40:30:30; zinc oxide, yellow iron oxide, red iron oxide and black iron oxide at a weight ratio of from 91:5:2:2 to 40:20:20:20; yellow iron oxide and red iron oxide at a weight ratio of from 90:10 to 50:50; and yellow iron oxide, red iron oxide and black iron oxide at a weight ratio of from 90:5:5 to 60:20:20.

4. The anti-UV composition of claim 1, wherein the fermented broth is mixed from 0.1 to 30.0 wt % relative to the Anti-UV composition for scalp.

5. The anti-UV composition of claim 1, wherein the fermented broth is manufactured by a method comprising:
(a) adding nuruk or yeast to the mixture comprising Houttuynia cordata, Perilla frutescens and Camellia sinensis and leaving the resultant as it is;
(b) adding ethanol to the resultant after the fermentation process in step (a);
(c) performing fermentation and curing at from 15 to 25° C.; and
(d) filtering the resultant obtained in step (c).

6. The anti-UV composition of claim 5, wherein, in step (1), Houttuynia cordata, Perilla frutescens and Camellia sinensis are mixed in the weight ratio of from 1 to 8:1 to 8:1 to 8.
